(19) United States
(12) Patent Application Publication
(10) Pub. No.: US 2004/0034336 A1
(43) Pub. Date: Feb. 19, 2004

(54) Charged liposomes/micelles with encapsulted medical compounds

(76) Inventors: Neal Scott, Houston, TX (US); Jerome Segal, Chevy Chase, CA (US)

Correspondence Address: MICHAEL E. KLICPERA, PO BOX 573, LA JOLLA, CA, 92038-0573, US

(21) Appl. No.: 10/214,959

(22) Filed: Aug. 8, 2002

(51) Int. Cl. A61K 39/395; A61K 31/727; A61K 9/127; A61K 9/22; A61K 38/06

(52) U.S. Cl. 604/890.1; 424/450; 514/18; 424/144.1; 514/56

(57) ABSTRACT

A charged liposome or micelle encapsulated therapeutic agent or medicament for the treatment of an obstruction in blood vessel. The present method comprises the steps of advancing a delivery catheter with a distal expansion member to the obstruction in a vessel, expanding the expansion member to a configuration wherein the expansion member dilates the obstruction and the expansion member delivers the charged liposomes or micelles with encapsulated therapeutic agents or medicaments to the obstruction. Electrical energy is applied to enhance tissue and cell penetration.

### BACKGROUND OF THE INVENTION

Cardiovascular disease is commonly accepted as being one of the most serious health risks facing our society today. Diseased and obstructed coronary arteries can restrict the flow of blood and cause tissue ischemia and necrosis. After over two decades of investigation, the exact etiology of sclerotic cardiovascular disease is still in question, the treatment of narrowed coronary arteries is more defined. Surgical construction of coronary artery bypass grafts (CABG) is often the method of choice when there are several diseased segments in one or multiple arteries. Open heart surgery is, of course, very traumatic for patients. In many cases, less traumatic, alternative methods are available for treating cardiovascular disease percutaneously. These alternate treatment methods generally employ various types of percutaneous transluminal angioplasty (PTCA) balloons or excising devices (atherectomy) to remodel or debulk diseased vessel segments. A further alternative treatment method involves percutaneous, intraluminal installation of expandable, tubular stents or prostheses in sclerotic lesions.

A recurrent problem with the previous devices and PTCA procedures is their failure to maintain patency due to the growth of injured vascular tissue. This is known as “restenosis” and may be a result of the original injury to the vessel wall occurring during the angioplasty procedure. Pathologically restenosis represents a neointimal proliferative response characterized by smooth muscle cell hyperplasia that results in reblockage of the vessel lumen necessitating repeat PTCA procedures up to 35-50% of all cases. It has been generally accepted that a certain therapeutic agents or medicaments may be capable of selectively inhibiting the growth of these hyperproliferating smooth muscle cells and thereby reduce the rate of restenosis after the primary interventional procedure.

Heretofore, various devices have been disclosed which may be used to deliver a therapeutic agent or medicament to a blood vessel while undergoing angioplasty. Balloon angioplasty catheters have been used to place and deliver a various therapeutic agents or medicaments within human vessels. For example, in U.S. Pat. Nos. 5,112,305, 5,746,716, 5,681,281, 5,873,852, 5,713,863 and 6,102,904 disclose and claim a balloon catheter system with various injector plates mounted on the balloon for delivering a drug into an arterial segment.

Alternatively a standard angioplasty balloon may be coated with a substrate or polymeric material which either incorporates, or is then used to bond, certain medicaments or theraputic agents. These agents are then delivered to the desired therapeutic site by inflation of the balloon and diffusion of the medicatment or therpeutic agent into the vessel wall. Only limited quantities of therapeutic agents can be delivered because of “wash-out” of the drug into the circulation during balloon placement and due to the limited time the inflated balloon can be left in place due to ischemia caused by the balloon.

In addition, previously disclosed methods of delivering drug to a site of treatment are described which utilize iontophoretic or electrophoretic means as disclosed in U.S. Pat. No. 5,499,971. Using these iontophoretic or electroporetic means passive diffusion of the drug or medicament is enhanced by placing the medicament or theraputic agent in close proximity to the site of treatment and then using electrical energy to augment delivery of the drug into the tissues or cells. These methods generally place the drug inside a balloon mounted distally on a catheter whereby the balloon is composed of a semi-porous material through which the drug can diffuse.

Additional devices have been disclosed which attempt to improve the depth of penetration into tissue by pressure driving a solution of the drug into the vessel wall through small orifices in the balloon material. There is, however, some evidence that high pressure “jetting” of a drug solution out of small pores close to the vessel lumen can in fact cause vessel wall injury. The development of double skinned, microporous (or weeping) balloons obviated this “jetting” effect to some extent, but diffusion of the drug into the vessel wall is still slow, and much of the drug can be lost through subsequent “washout effects”. This method leads to limited amounts of drugs or therapeutics agents delivered to the tissues or cells. Furthermore, in all of these methods the balloon must be expanded and thereby restricts blood flow to the distal arterial segments while the balloon is in the expanded configuration thus limiting the time the drug delivering balloon can be clinically utilized.

There are also several disadvantages using either a stent or balloon catheter to delivery a therapeutic agent or medicament to a vascular segment. Regarding the therapeutic agent eluting stents, once the stent is deployed, there is no means outside of invasive surgical excision, to remove the eluting stent from the vascular segment. Therefore, stents or implanted prostheses with therapeutic agent eluting properties must be precisely calibrated to deliver an exact quantity of the therapeutic agent or medicament to the vascular segment upon stent deployment. Balloon catheters employed to delivery a therapeutic agent or medicament to a vascular segment have limitations including potential balloon rupture and ischemia due to balloon inflation limiting distal blood flow to the artery. This leads to tissue ischemia and potential necrosis. Even “perfusion” type angioplasty balloons used to delivery a therapeutic agent or medicament to the affected artery provide far less than physiological blood flow during balloon inflation and dwell times are limited by ischemia and tissue necrosis.

Recent studies have demonstrated the effectiveness of a number of agents (e.g., paclitaxel, rapamycin, Actinomycin D) on the prevention of unwanted cellular proliferation. These agents have proven efficacy in the treatment of cancer and transplant rejection. A major advantage of these agents is the high lipid solubility that causes tissue levels to be high for an extended period of time since they cannot be rapidly cleared. However, this advantage is also a disadvantage because the delivery of these medicaments must generally pass hydrophilic boundaries.

Thus, it can be seen that there is a need for a new and improved apparatus and method to selectively delivery a therapeutic agent or medicament to an arterial segment and which overcomes these disadvantages.

In general, it is an object of this present invention to provide an electrically charged liposome or micelle encapsulating a medicament and method which is capable of delivering, by an active means, the liposome or micelle encapsulated therapeutic agent or medicament to the vessel segment or obstruction.

In general, it is an object of this present invention to provide an electrically charged liposome or micelle encapsulating a medicament and method which is capable of delivering, by an electrical means, the liposome or micelle encapsulated therapeutic agent or medicament to the vessel segment or obstruction.

Another object of the invention is to provide a method to deliver high concentrations of agents that are poorly soluble or insoluble in aqueous media to selected sites in the body including arteries, veins or other tubular structures, prosthetic devices such as grafts, and tissues such as, but not limited to, brain, myocardium, colon, liver, breast and lung.

Another object of the invention is to provide a apparatus that can control the release or diffusion of a medicament or therapeutic agent to minimize potential systemic affects and maximize the diffusion or delivery of the medicament or therapeutic agent to the site of treatment.

### SUMMARY OF THE INVENTION

It is known that therapeutic agent therapy can reduce the proliferation of rapidly growing cells. The present invention comprises an electrically charged liposome or micelle that encapsulates a therapeutic agent or medicament. In addition, the methods necessary for deployment and delivery of the charged liposomes or micelles encapsulating a therapeutic agent or medicament to an obstruction in a vessel are also disclosed and claimed.

Since the therapeutic agent or medicament is capable of selectively inhibiting the growth of proliferating cells, the present invention not only achieves acute patency of a vessel but employs medical therapy to maintain chronic patency through the prevention of restenosis.

The invention also takes advantage of the prior body of knowledge that has demonstrated the enhanced solubility and delivery of agents after they have been incorporated into liposome or micelles or micelles. Since liposome or micelles and micelles possess both lipophilic and hydrophilic regions, they can be used to solubilize compounds that are insoluble in water. Electrically charging the liposome or micelles can facilitate the movement of the charged liposome or micelle in an electrical field.

This disclosure also demonstrates the delivery of charged, lipophilic medicaments or agents by incorporating them into charged liposome or micelles and then delivering them to the target site by electrophoresis.

The delivering of the charged present invention method also comprises the steps of advancing a catheter generally including a distal expansion member and advancing it to the obstruction in a vessel. At this time the clinician applies forces on the expansion member causing the expansion member to become fully expanded wherein the expansion member dilates the obstruction. Then a means is employed which actively delivers the liposome or micelle-encapsulated therapeutic agent or medicament to the obstruction or vessel wall.

One approach may be to 1) energize a delivery catheter to create a bond between the charged liposome or micelle encapsulating the therapeutic agent and the distal expansion means, 2) advance the system to the treatment segment, 3) expand the expansion member to dilate the segment, 4) apply electrical energy to cause iontophoresis of the therapeutic agent into the tissues and/or liposome or micelle encapsulating the therapeutic agent 5) apply electrical energy for electroporation to be applied to permeabilize the cells. Preferably, the catheter is able to perform steps 3, 4 and 5 sequentially without repositioning of the catheter. Even more preferably, the catheter is designed to maintain a high concentration of drug in the tissue extracellular spaces (e.g. by iontophoresis) such that the subsequent creation of transient pores in cell surface membranes by electroporation pulses results in greatly improved intracellular delivery of the medicament or therapeutic agent.

Another approach may be to 1) prepare a delivery catheter to inject charged liposomes or micelles encapsulating the therapeutic agent through the distal expansion means, 2) advance the system to the treatment segment, 3) expand the expansion member to dilate the segment, 4) inject the charged liposomes or micelles encapsulating the therapeutic agent 5) apply electrical energy to cause iontophoresis of the therapeutic agent into the tissues and/or 6) apply electrical energy for electroporation to be applied to permeabilize the cells. Preferably, the catheter is able to perform steps 3, 4 and 5 and 6 sequentially without repositioning of the catheter. Even more preferably, the catheter is designed to maintain a high concentration of drug in the tissue extracellular spaces (e.g. by iontophoresis) such that the subsequent creation of transient pores in cell surface membranes by electroporation pulses results in greatly improved intracellular delivery of the medicament or therapeutic agent.

### BRIEF DESCRIPTION OF THE DRAWINGS

FIG. 1 is a cross-sectional view of a charged micelle structure encapsulating a therapeutic agent.

FIG. 2 is a cross-sectional view of a charged liposome structure encapsulating a therapeutic agent.

FIG. 3 is a cross-sectional view taken along the line 2-2 of FIG. 2.

FIG. 4 is a representation of the present invention micelle encapsulating a therapeutic agent and having an overall positive charge.

FIG. 5 is a representation of the present invention liposome encapsulating a therapeutic agent and having an overall positive charge.

FIG. 6 is a representation of the present invention micelle encapsulating a therapeutic agent and having an overall negative charge.

FIG. 7 is a representation of the present invention liposome encapsulating a therapeutic agent and having an overall negative charge.

### DETAILED DESCRIPTION OF THE DRAWINGS

In general, the present invention relates generally to devices and methods that are used to deliver a medicament or therapeutic agent to an obstruction within a stenotic segment of a vessel. The present invention is comprised of a lipsosome or micelle structure that encapsulates a medicament or therapeutic agent and has an overall electrical charge

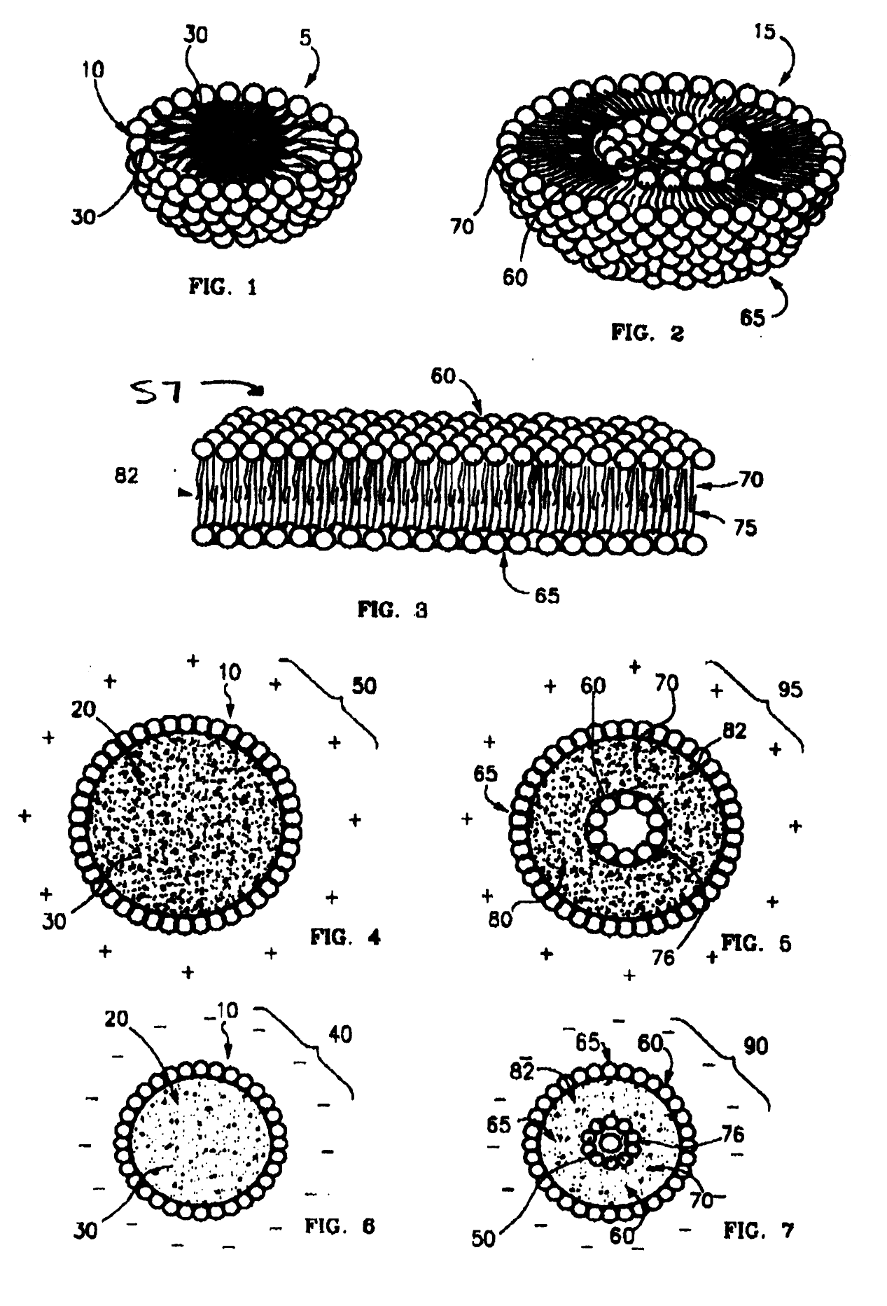

As shown in FIGS. 1, 4 and 6, the present invention micelle generally comprises a plurality of outer hydrophilic heads 10 that encapsulate a plurality of inner hydrophobic tails 20. Therapeutic agents or medicaments 30 with hydrophobic characteristics can be incorporated within the inner hydrophobic tail region 25. To function as the present invention, this micelle/medicament composite will include an overall negative 40 or positive charge 50.

As shown in FIGS. 2, 3, 5 and 7, present invention liposomes generally comprise a bi-layer or double structure 57. Shown more specifically in FIG. 3, the hydrophilic heads 65 of the molecules are on the outside of the bi-layer, and the hydrophobic tails 75 point toward the interior of the bi-layer. In the spherical structures shown in FIGS. 2, 5, and 7, there are two inner regions, an first inner hydrophobic tail region 77 surrounding another inner hydrophilic tail region 76. Therapeutic agents or medicaments 80 with hydrophobic characteristics can be incorporated within the inner hydrophobic region 76. Whereas therapeutic agents or medicaments 82 with hydrophilic characteristics can be incorporate within the second inner hydrophilic region 77. It is not essential that the therapeutic agents or medicaments be common between the two regions, inner hydrophobic tail region 77 can contain a therapeutic agent different from that of inner hydrophobic tail region 76.

The liposome-encapsulated therapeutic agent, 80 or 82, or micelle encapsulated therapeutic agent 30, can be an anticoagulant selected from the group consisting of D-Phe-Pro-Arg chloromethyl ketone, an RGD peptide-containing compound, heparin, an anti-thrombin compound, a platelet receptor antagonist, an anti-thrombin antibody, an anti-platelet receptor antibody, hirudin, hirulog, phe-pro-arg-chloromethyketone (Ppack), Factor VIIa, Factor Xa, aspirin, clopridogrel, ticlopidine, a prostaglandin inhibitor, a platelet inhibitor and a tick anti-platelet peptide, and combinations thereof.

The liposome-encapsulated therapeutic agent 80, 82 or micelle-encapsulated therapeutic agent 30, can be a promoter of vascular cell growth, such as a growth factor stimulator, a growth factor receptor agonist, a transcriptional activator, and a translational promoter.

Alternatively, the therapeutic agents can be an inhibitor of vascular cell growth, selected from the group consisting of a growth factor inhibitor, a growth factor receptor antagonist, a transcriptional repressor, a translational repressor, an antisense DNA, an antisense RNA, synthetic DNA compounds, especially those with backbones that have been modified to inhibit enzymatic degradation (e.g. phosphorothioate compounds and morpholino diamidate compounds), a replication inhibitor, an inhibitory antibody, an antibody directed against growth factors, a bifunctional molecule consisting of a growth factor and a cytotoxin, and a bifunctional molecule consisting of an antibody and a cytotoxin, double stranded DNA, single stranded DNA, single stranded RNA and a double stranded RNA and combinations thereof.

The liposome-encapsulated therapeutic agent 80, 82 or micelle-encapsulated therapeutic agent 30, can be selected from the group consisting of a cholesterol-lowering agent, a vasodilating agent, and agents which interfere with endogenous vasoactive mechanisms, estrogen, testosterone, steroid hormones, cortisol, dexamethasone, corticosteroids, thyroid hormones, thyroid hormones analogs, throid hormones antagonist, adrenocorticotrophic hormone, thyroid stimulating hormone, thyroid releasing factor, thyroid releasing factor analogs, thyroid releasing factor antagonists and combinations thereof.

Additionally, the therapeutic agents 30, 80, or 82 can be smooth muscle inhibitor, such as a selected from the group consisting of an agent that modulates intracellular calcium binding proteins, a receptor blocker for contractile agonists, an inhibitor of the sodium/hydrogen antiporter, a protease inhibitor, a nitrovasodilator, a phosphodiesterase inhibitor, a phenothiazine, a growth factor receptor agonist, an anti-mitotic agent, an immunosuppressive agent, and a protein kinase inhibitor, and combinations thereof.

In addition, the therapeutic agents 30, 80 and 82 can be a compound that inhibits cellular proliferation, Paclitaxel, Rapamycin, Actinomycin D, Methotrexate, Doxorubicin, cyclophosphamide, and 5-fluorouracil, 6-mercapatopurine, 6-thioguanine, cytoxan, cytarabinoside, cis-platin, chlorambucil, busulfan, and any other drug that can inhibit cell proliferation, and combinations thereof.

The charged liposome-encapsulating a medicament or therapeutic agent 15 or micelle-encapsulated a medicament or therapeutic agent 5 may be disposed on or within a substrate or polymer 43, which can be biodegradable and adapted for slow release of the liposome or micelle-encapsulated therapeutic agents 30, 80, or 82. A substrate or polymer 43 laden with one or more therapeutic agents 30, 80, or 82 can be positioned on the surface of a balloon or alternately injected through a delivery catheter.

A biodegradable substrate or polymer 43 such as polylactide, polyanhydride, polyorthoester or polyglycolide, for example can be used. In addition to synthetic polymers, natural polymers can be used, such as amino acid polymers or polysaccharides. The polymer or substrate 43 is selected depending on the charged liposome-encapsulating a medicament or therapeutic agent 15 or micelle-encapsulated a medicament or therapeutic agent 5 used. In addition, the substrates or polymers 43 compatibility with a patient and the ultimate pharmacologic effect are desired. For example, if the effect needs to only last a short period, that a thin polymer 43. Alternatively, only the layer closest to the body fluid would contain the charged liposome-encapsulating a medicament or therapeutic agent 15 or micelle-encapsulated a medicament or therapeutic agent 5. Another alternative would be to use a polymer 43 which is biodegradable over a long period of time. Naturally, the other characteristics would be selected for a desired prolonged release.

A plurality of charged liposomes-encapsulating a medicament or therapeutic agent 15 or micelles-encapsulated a medicament or therapeutic agent 5 can be coated on (or incorporated into a polymer or other substrate 43 and coated on the expansion means or balloon distally mounted on a catheter. Or a plurality of charged liposomes-encapsulating a medicament or therapeutic agent 15 or micelles-encapsulated a medicament or therapeutic agent 5 can be delivered to a treatment site by an injection delivery device or pressure mediated catheter. The apparatuses for delivering or infusing a therapeutic agent or medicament is known to those skilled art or can be determined by reference to standard references.

Once the site of obstruction or treatment is reached, a charge could be applied or reversed thus driving the plurality of charged liposomes-encapsulating a medicament or therapeutic agent 15 or micelles-encapsulated a medicament or therapeutic agent 5 into the target tissue. In this case, the electrode placed on the skin of the patient would be used to cause active diffusion or iontophoresis of the therapeutic agent or medicament into the target tissues. The present invention can benefit from the flow of electrical current in the form of various waveforms to perform the iontophoresis and/or electroporation procedures. Possible waveforms contemplated for the present invention include square waves, rectangular waves, saw-toothed waves, sinusoidal waves that do not reverse polarity, rectified sinusoidal waves, and other waveform shapes which may reverse polarity but provide a net flow of current in the desired direction.

Electrical current could also be coordinated with the patient's elctrocardiogram such that electrical current is provided to the mesh only during certain phases of cardiac depolarization. This “gating” of the electrical current would avoid the potential danger of discharging electrical current to the heart during vunerable phases of depolarization which may lead to cardiac arrhythmias.

Iontophoretically enhanced delivery requires that the therapeutic agent carry a net charge under physiological conditions whereas electroporation alone would be used for delivering treatment agents that are not sufficiently ionized to iontophorese well into tissues. Electroporation may also be the preferred strategy for enhancing localized cellular targeting of a systemically administered therapeutic agent.

As used herein, the term “iontophoresis” means the migration of ionizable molecules through a medium driven by an applied low-level electrical potential. This electrically mediated movement of molecules into tissues is superimposed upon concentration gradient dependent diffusion processes. If the medium or tissue through which the molecules travel also carries a charge, some electro-osmotic flow occurs. However, generally, the rate of migration of molecules with a net negative charge towards the positive electrode and vice versa is determined by the net charge on the moving molecules and the applied electrical potential. The driving force may also be considered as electrostatic repulsion. Iontophoresis usually requires relatively low constant DC current in the range of from about 2-10 mA. In a well established application of iontophoresis, that of enhancing drug delivery through the skin (transdermal iontophoresis), one electrode is positioned over the treatment area and the second electrode is located at a remote site, usually somewhere else on the skin. With the present invention the return electrode may be similarly positioned on the skin. Alternatively the tip of the guide wire emerging from the distal end of the support catheter may serve as the return electrode.

As used herein, the term “electroporation” means the temporary creation of holes or aqueous pores in the surface of a cell membrane by an applied electrical potential and through which therapeutic agents may pass into the cell. Electroporation is now widely used in biology, particularly for transfection studies, where plasmids, DNA fragments and other genetic material are introduced into living cells. During electroporation pulsing, molecules that are not normally membrane permeant are able to pass from the extracellular environment into the cells during the period of induced reversible membrane permeabilization. The permeabilized state is caused by the generation of an electrical field in the cell suspension or tissue of sufficient field strength to perturb the cell surface membrane's proteolipid structure. This perturbation (sometimes referred to as dielectric breakdown) is believed to be due to both a constituent charge separation and the effect of viscoelastic compression forces within the membrane and it's sub-adjacent cytoskeletal structures. The result is a localized membrane thinning. At a critical external field strength, pores or small domains of increased permeability are formed in the membrane proteolipid bi-layer.

Operation and use of a general delivery of the present invention may now be briefly described as follows. Let it be assumed that the patient which the medical procedure is to be performed using a medicament delivery device to treat one or more stenoses which at least partially occlude one or more arterial vessels, and it is desirable to enlarge the flow passages through the stenoses. Typically the drug delivery device would be supplied by the manufacturer with the expansion member in its most contracted or deflated position. The expansion member could be coated or the injection catheter prepared with the present inventions to provide a means of transfer to the vessel wall. In the coating example, a container having a solution of the charged liposomes or micelles encapsulating therapeutic agents 5 or 15, can be separately supplied whereby sometime prior to inserting the mechanical dilatation and medicament delivery device into the patient, the expansion member is immersed or dipped into the container in order to coat the expansion member with the present invention. Appropriate time and/or temperatures will be allowed for the medicament solution to adsorb, dry and adhere to the polymer coated expansion mesh, or alternately, a charge can be applied to facilitate bonding of the medicament or therapeutic agent to the polymer coated expansion member.

Alternately, the drug delivery device can have a means, such a series of injector plates or pores in the expansion member, to inject or infuse the present invention charged liposome or micelle with encapsulated medicaments 5, 15 into the vessel wall of the treatment site. If this type of medicament delivery catheter is used, a solution of charged liposomes or micelles encapsulating therapeutic agents 5 or 15, can be supplied whereby sometime prior to inserting the medicament delivery device into the patient, the catheter is first prepared according to standard procedures.

The delivery device is then inserted into a guiding catheter (not shown) typically used in such a procedure and introduced into the femoral artery and having its distal extremity in engagement with the ostium of the selected coronary artery.

It is desirable, more importantly, in the coated delivery catheter or means is used to deliver the present invention to a treatment site, that they have the capability to apply an electrical current with a charge opposite to that of the therapeutic agent or medicament encapsulated liposome or micelle 5 or 15. When the present invention liposome or micelle-encapsulated therapeutic agents or medicaments 5, 15 have an inherent charge potentials, a charge opposite that can be applied by, for example, the expansion member. This results in an electrical bond established between the surface of the expansion member and the liposome or micelle-encapsulated therapeutic agent or medicament 5, 15. The continuously charged expansion member with the attached charged liposome or micelle-encapsulated therapeutic agent or medicament 5, 15 could then be advanced through the patient's vasculature to the site of dilatation and therapy without significant loss of the medicament in the bloodstream.

The medicament delivery device is then advanced in a conventional manner by the physician undertaking the procedure and into the vessel containing a stenosis. Generally, once positioned within the stenosis, the expansion member is expanded with the charged liposome or micelle-encapsulated medicament or therapeutic agent 5, 15 coated thereo. Alternately, when using the injection type medicament deliver device, expansion of the distal member provides proper orientation for injection into the vessel of the charged liposomes or micelles encapsulating a medicament or therapeutic agent 5, 15. Generally, a means separate from the expansion means, will than be employed to cause injection or infusion of the charged liposomes or micelles encapsulating a medicament or therapeutic agent 5, 15 into the vessel wall or obstruction.

After the expansion member is expanded and the obstruction dilated, or the present invention injected or infused into the lesion, an electrical charge is provided to the expansion member or other means that is in close proximity to the liposomes or micelles encapsulating the medicaments or therapeutic agents. This electrical charge is opposite to the overall charge of the liposomes or micelles encapsulating the medicaments or therapeutics agents 5, 15 or alternately, the charge used to bind the liposomes or micelles encapsulating the medicament 5, 15 to the expansion member. This charge will then tend to drive the liposomes or micelles encapsulated medicament or therapeutic agent 5, 15 into the tissue through iontophoretic means. The iontophoretic process is known to facilitate or assist the transport of the liposomes or micelles with encapsulated medicaments or therapeutic agents 5, 15 across the selectively permeable membranes and enhance tissue penetration. Since the present invention involves the use of electrical energy, there are many possible waveforms contemplated for use, square waves, rectangular waves, saw toothed waves, sinusoidal waves that do not reverse polarity, rectified sinusoidal waves, and modified rectangular or other waves, that can be employed. The primary characteristic of the preferred waveforms is that they all provide a net flow of current to urge the liposomes or micelles encapsulating the medicaments or therapeutics agents into the cell membranes. It must be appreciated by those skilled in the art, that the waveforms with frequencies and duty cycles must be capable of delivering the desired current under varying impedances encountered by the expansion member and the surrounding vessel wall and fluids.

After a predetermine time, the electrical current can be altered to achieve another purpose or terminated. This makes it possible to maintain dilatation and medicament delivery of the obstruction over extended periods of time when desired.

After dilatation and delivery of the liposomes or micelles encapsulating the medicaments or therapeutics agents 5, 15 to the lesion has been carried out for an appropriate length of time, the expansion member can be changed from its expanded position to a contracted position and can be removed along with the guide wire after which the guiding catheter (not shown) can be removed and the puncture site leading to the femoral artery closed in a conventional manner.

Although, the procedure hereinbefore described was for treatment of a single stenosis, it should be appreciated that if desired during the same time that the delivery device can be re-loaded with the present invention liposomes or micelles then other vessels of the patient having stenoses therein can be treated in a similar manner.

Describe below are some examples of experiments conducted using the present invention.

### EXAMPLE 1

### Local Delivery of 7-Amino Actinomycin D

7-Amino Actinomycin D is a fluorescent (emits at 610 nm, [red]) analog of Actinomycin D, a potent inhibitor of cellular proliferation. It is very lipophilic and poorly soluble in water. Liposome or micelles were prepared by mixing 3.0 mg of phosphatidylcholine, 3.0 mg of cholesterol and 0.3 mg of phosphatidylserine in a test tube. Chloroform (200 microliters) was added and the solution was evaporated to dryness in a test tube. 7-Amino Actinomycin D (500 mg) was dissolved in 8 mM CaCl₂ for a final concentration of 0.5 mg/ml. The 7-Amino Actinomycin D solution was added to the lipid mixture in small aliquots with constant stirring. The hydrogel-coated metal mesh catheter was placed in the 7-amino Actinomycin D/liposome or micelle mixture and then used for drug delivery in the following manner: The hydrogel-coated metal mesh catheter was placed in the 7-Amino Actinomycin D/liposome or micelle mixture and then removed. In some cases, the hydrogel-coated mesh portion of the catheter was covered with a retractable sheath to prevent loss of the compound during the transport of the catheter from the arterial access site to the target site. When the catheter was positioned at the target site the sheath was retracted and the mesh was expanded against the arterial wall. Iontophoersis was performed by applying an electrical current to the mesh. The circuit was completed by pacing a patch on the skin that was connected to the circuit and had an opposite charge than the mesh. In this example the iontophoresis parameters were 5 mA, and 8 V, applied for 10 minutes. The results also show 7-Amino Actinomycin D throughout the vessel wall and in the outer layer of the vessel. There is also evidence of localization of the 7-Amino Actinomycin D in the nuclei of the cells.

### EXAMPLE 2

### Local Delivery of Paclitaxel

Paclitaxel is one of the most potent inhibitors of cellular proliferation in clinical use and has been shown to be efficacious in a large number of cancers. Paclitaxel is very lipophilic and essentially insoluble in water. Liposome or micelles were prepared by mixing 0.72 mg phosphatidylcholine and 0.8 mg of phosphatidylserine in a test tube with 800 microliters of chloroform. The solution was evaporated to dryness. Paclitaxel labeled with a fluorescent probe (Oregon Green) was dissolved in methanol to obtain a 201 mg/1 ml solution. Twenty-five microliters of this solution was combined with 975 microliters of 8 mM CaCl₂. The paclitaxel solution was added to the dried lipid mixture in small aliquots with constant stirring. The hydrogel-coated metal mesh catheter was placed in the paclitaxel/liposome or micelle mixture and then removed. In some cases, the hydrogel-coated mesh portion of the catheter is covered with a retractable sheath to prevent loss of the compound during the transport of the catheter from the arterial access site to the target site. When the catheter was positioned at the target site the sheath was retracted and the mesh was expanded against the arterial wall. Iontophoersis was performed by applying an electrical current to the mesh. The circuit was completed by pacing a patch on the skin that was connected to the circuit and had an opposite charge than the mesh. In this example the iontophoresis parameters were 7 mA and 8 V, applied for 20 minutes. The results showed the paclitaxel throughout the vessel wall and in the outer layer of the vessel.

We claim:

1. An apparatus for delivering a medicament to an obstruction within a vascular segment or a body passageway which comprises:
an electrically charged liposome or micelle encapsulated a therapeutic agent or medicament.

2. An apparatus as recited in claim 1 wherein said apparatus has a negative charge.

3. An apparatus as recited in claim 1 wherein said apparatus has a positive charge.

4. An apparatus as recited in claim 1, wherein said charged liposome or micelle encapsulating a therapeutic agent or medicament will function to migrate by iontophoretic means into target tissues of said vascular segment.

5. An apparatus as recited in claim 1, wherein said charged liposome or micelle encapsulating a therapeutic agent or medicament will function to migrate by electroporation means into target tissues of said vascular segment.

6. An apparatus as recited in claim 1, wherein said liposome or micelle-encapsulated agent or medicament is an anticoagulant selected from the group consisting of D-Phe-Pro-Arg chloromethyl ketone, an RGD peptide-containing compound, heparin, an anti-thrombin compound, a platelet receptor antagonist, an anti-thrombin antibody, an anti-platelet receptor antibody, hirudin, hirulog, phe-pro-arg-chloromethyketone (Ppack), Factor VIIa, Factor Xa, aspirin, clopridogrel, ticlopidine, a prostaglandin inhibitor, a platelet inhibitor and a tick anti-platelet peptide, and combinations thereof.

7. An apparatus as recited in claim 1, wherein said liposome or micelle-encapsulated agent or medicament is a promoter of vascular cell growth selected from the group consisting of a growth factor stimulator, a growth factor receptor.

8. An apparatus as recited in claim 1, wherein said liposome or micelle-encapsulated agent or medicament is an inhibitor of vascular cell growth selected from the group consisting of a growth factor inhibitor, a growth factor receptor antagonist, a transcriptional repressor, a translational repressor, an antisense DNA, an antisense RNA, synthetic DNA compounds, especially those with backbones that have been modified to inhibit enzymatic degradation (e.g. phosphorothioate compounds and morpholino diamidate compounds), a replication inhibitor, an inhibitory antibody, an antibody directed against growth factors, a bifunctional molecule consisting of a growth factor and a cytotoxin, and a bifunctional molecule consisting of an antibody and a cytotoxin, double stranded DNA, single stranded DNA, single stranded RNA and a double stranded RNA and combinations thereof.

9. An apparatus as recited in claim 1, wherein said liposome or micelle-encapsulated agent or medicament is selected from the group consisting of a cholesterol-lowering agent, a vasodilating agent, and agents which interfere with endogenous vasoactive mechanisms, estrogen, testosterone, steroid hormones, cortisol, dexamethasone, corticosteroids, thyroid hormones, thyroid hormones analogs, throid hormones antagonist, adrenocorticotrophic hormone, thyroid stimulating hormone, thyroid releasing factor, thyroid releasing factor analogs, thyroid releasing factor antagonists and combinations thereof.

10. An apparatus as recited in claim 1, wherein said liposome or micelle-encapsulated agent or medicament is a smooth muscle inhibitor selected from the group consisting of an agent that modulates intracellular calcium binding proteins, a receptor blocker for contractile agonists, an inhibitor of the sodium/hydrogen antiporter, a protease inhibitor, a nitrovasodilator, a phosphodiesterase inhibitor, a phenothiazine, a growth factor receptor agonist, an anti-mitotic agent, an immunosuppressive agent, and a protein kinase inhibitor, and combinations thereof.

11. An apparatus as recited in claim 1, wherein said liposome or micelle-encapsulated agent or medicament is a compound that inhibits cellular proliferation, Paclitaxel, Rapamycin, Actinomycin D, Methotrexate, Doxorubicin, cyclophosphamide, and 5-fluorouracil, 6-mercapatopurine, 6-thioguanine, cytoxan, cytarabinoside, cis-platin, chlorambucil, busulfan, and any other drug that can inhibit cell proliferation, and combinations thereof.

12. An apparatus as recited in claim 1, wherein said liposome or micelle-encapsulated agent or medicament will migrate into target tissues when exposed to an electrical energy applied by an electrical delivery device.

13. An apparatus as recited in claim 1, wherein said charged liposome or micelle-encapsulated agent or medicament will iontophoretical transfer into tissues of said vascular segment when exposed to an electrical energy applied by an electrical delivery catheter.

14. An apparatus as recited in claim 1, wherein said charged liposome or micelle-encapsulated agent or medicament will at least partially electroporation transfer into target tissues of said vascular segment when exposed to an electrical energy applied by an electrical delivery catheter.

15. An apparatus for delivering a medicament to an obstruction within a vascular segment or a body passageway which comprises:
an plurality of electrically charged liposomes or micelles encapsulating a therapeutic agent or medicament;
said charged liposomes of micelles having the function to be at least partially infused into target tissues.

16. An apparatus as recited in claim 15, wherein said liposome or micelle-encapsulated agent or medicament is an anticoagulant selected from the group consisting of D-Phe-Pro-Arg chloromethyl ketone, an RGD peptide-containing compound, heparin, an antithrombin compound, a platelet receptor antagonist, an anti-thrombin antibody, an anti-platelet receptor antibody, hirudin, hirulog, phe-pro-arg-chloromethyketone (Ppack), Factor VIIa, Factor Xa, aspirin, clopridogrel, ticlopidine, a prostaglandin inhibitor, a platelet inhibitor and a tick anti-platelet peptide, and combinations thereof.

17. An apparatus as recited in claim 15, wherein said liposome or micelle-encapsulated agent or medicament is a promoter of vascular cell growth selected from the group consisting of a growth factor stimulator, a growth factor receptor.

18. An apparatus as recited in claim 15, wherein said liposome or micelle-encapsulated agent or medicament is an inhibitor of vascular cell growth selected from the group consisting of a growth factor inhibitor, a growth factor receptor antagonist, a transcriptional repressor, a translational repressor, an antisense DNA, an antisense RNA, synthetic DNA compounds, especially those with backbones that have been modified to inhibit enzymatic degradation (e.g. phosphorothioate compounds and morpholino diamidate compounds), a replication inhibitor, an inhibitory antibody, an antibody directed against growth factors, a bifunctional molecule consisting of a growth factor and a cytotoxin, and a bifunctional molecule consisting of an antibody and a cytotoxin, double stranded DNA, single stranded DNA, single stranded RNA and a double stranded RNA and combinations thereof.

19. An apparatus as recited in claim 15, wherein said liposome or micelle-encapsulated agent or medicament is selected from the group consisting of a cholesterol-lowering agent, a vasodilating agent, and agents which interfere with endogenous vasoactive mechanisms, estrogen, testosterone, steroid hormones, cortisol, dexamethasone, corticosteroids, thyroid hormones, thyroid hormones analogs, throid hormones antagonist, adrenocorticotrophic hormone, thyroid stimulating hormone, thyroid releasing factor, thyroid releasing factor analogs, thyroid releasing factor antagonists and combinations thereof.

20. An apparatus as recited in claim 15, wherein said liposome or micelle-encapsulated agent or medicament is a smooth muscle inhibitor selected from the group consisting of an agent that modulates intracellular calcium binding proteins, a receptor blocker for contractile agonists, an inhibitor of the sodium/hydrogen antiporter, a protease inhibitor, a nitrovasodilator, a phosphodiesterase inhibitor, a phenothiazine, a growth factor receptor agonist, an anti-mitotic agent, an immunosuppressive agent, and a protein kinase inhibitor, and combinations thereof.

21. An apparatus as recited in claim 15, wherein said liposome or micelle-encapsulated agent or medicament is a compound that inhibits cellular proliferation, Paclitaxel, Rapamycin, Actinomycin D, Methotrexate, Doxorubicin, cyclophosphamide, and 5-fluorouracil, 6-mercapatopurine, 6-thioguanine, cytoxan, cytarabinoside, cis-platin, chlorambucil, busulfan, and any other drug that can inhibit cell proliferation, and combinations thereof.

22. A method for introducing charged liposomal encapsulated or micelle-encapsulated medicaments into cells of a patient, comprising the steps of:
Selecting a catheter with a distal delivery member wherein a portion of said delivery member contacts the vessel wall at a predetermine location;
applying a predetermined electric signal to said catheter to assist in transporting said liposome or micelle-encapsulated medicaments across cell membranes.

23. A method for simultaneously performing coronary angioplasty and delivering a charged liposome or micelle containing an encapsulated agent or medicament to a localized area of a passageway with a delivery catheter, method comprising the steps of:
a) advancing the delivery catheter with a distal expansion member through the passageway until the expansion member is adjacent to the localized area;
b) employing a means to expand the expansion member and apply a pressure against the localized area of the passageway thereby dilating the localized area of the passageway; and
c) phoretically transporting the charged liposome or micelle encapsulating an therapeutic agent or medicament to the localized area.

24. An in vivo method of introducing molecules into cells of a patient for therapeutic purposes, comprising the steps of:
providing a catheter means having an expandable distal portion and a means for generating an electric field;
locating the distal portion of the catheter to a selected location into a selected blood vessel of the patient,
expanding the distal portion of the catheter for dilating an obstruction within the blood vessel, and
delivering charged liposomes or micelles with encapsulated agent or medicament to the obstruction.
